(19) United States
(12) Patent Application Publication
(10) Pub. No.: US 2006/0078628 A1
(43) Pub. Date: Apr. 13, 2006

(54) Wound treating agent

(76) Inventors: Karl Koman, Salzburg (AT); Claudia Arnold, Balzheim (DE); Urs Ruegger, Zofingen (CH)

Correspondence Address: Harness Dickey and Pierce, PLC, P.O. Box 828, Bloomfield Hills, MI, 48303, US

(21) Appl. No.: 11/205,912

(22) Filed: Aug. 17, 2005

Related U.S. Application Data
(60) Provisional application No. 60/648,611, filed on Jan. 31, 2005.

(30) Foreign Application Priority Data
Oct. 9, 2004 (EP) 04 024 140.8

(51) Int. Cl. A61K 33/36

(52) U.S. Cl. 424/672

(57) ABSTRACT

The invention relates to a wound treating agent for humans and/or animals which contains a zeolite-iodine inclusion compound, to the use of such wound treating agent for the preparation of wound-stilling materials, especially compresses, plasters and/or dressing material, and to such wound-stilling materials.

### CROSS-REFERENCE TO RELATED APPLICATIONS

This application claims the benefit of U.S. Provisional Application No. 60/648,611, filed on Jan. 31, 2005 which claims the benefit of EP 04 024 140.8, filed Oct. 9, 2004. The disclosure(s) of the above application(s) is (are) incorporated herein by reference.

### FIELD OF THE INVENTION

The present invention relates to a wound treating agent for humans and animals, to its use for the preparation of wound-stilling materials, and to such wound-stilling materials.

### BACKGROUND OF THE INVENTION

Elemental iodine has long been used for the disinfection of wounds. It is an efficient microbicide which does not cause any resistances and promotes rapid wound healing without inflammation or local or systemic infection due to the disinfection of the wound. Elemental iodine as such cannot be employed; it requires formulation, for example, as an alcoholic iodine tincture.

Iodine is preferably used in connection with an iodophor. An iodophor is a support for iodine which slowly releases the active substance and thus sustains its activity and, in addition, keeps the local concentration low and thus improves effectiveness. A typical iodophor is polyvinylpyrrolidone. However, to apply these formulations, it is necessary that the blood flow is first stopped in order that the iodine can get to the wound surface. For hemostasis in large-area or deep wounds, a wide variety of compounds are used whose effect is mostly to precipitate proteins from the blood and thus to accelerate the formation of scab, or else to bind moisture and to become agglomerated with the blood to form a wound closure. These agents may be traditional, such as coffee meal, or they may contain metal salts, such as aluminum hydroxide, or biochemically active substances which directly interfere with the process of blood coagulation. A known agent is zeolite, which is characterized by a particularly quick onset of action in hemostasis. EP 0 888 783 B1 describes the use of zeolites for hemostasis and promoting the wound healing.

Zeolites are characterized by the ability to adsorb up to 25% of water, based on their mass. In this way, they can provide for concentration of the coagulation factors and for a quick coagulation of the blood.

### SUMMARY OF THE INVENTION

U.S. Pat. No. 4,822,349 describes such a method for hemostasis in warm-blooded mammals including humans in which a sterile dehydrated zeolite is applied to the wound directly or by means of a dressing, so that the molecular sieve causes the coagulation effect by concentration. RU 2143908 C1 describes the application of zeolite-containing tuff, and RU 2185149 C1 describes the use of liquid or powdery zeolite-containing formulations for the treatment of skin lesions.

One peculiarity of zeolite is the fact that it need not be mixed directly with the blood to display its activity. WO 02/30479 describes the use of zeolite in non-woven packages for accelerating coagulation. Moisture is absorbed even if the zeolite is, not too distantly, separated from the pool of liquid, for example, through a non-woven material which is provided between the zeolite and the wound. A particular advantage is that the wound does not become crusted by the mineral foreign matter in this way, but remains clean.

WO 02/22060 A1 describes compresses for wound treatment which contain a zeolitic filler material, this zeolitic filler material being permanently loaded with antimicrobial silver. Although blood coagulation and a disinfecting effect are achieved thereby, the water-absorbing effect and thus the acceleration of coagulation is subject to some need of improvement due to the permanent silver loading of the zeolite, and for a disinfecting effect, an actual or almost direct contact with the wound is necessary, which bears the risk of damaging healthy cells.

US 2001/0009831 A1 also describes the use of permanently silver-loaded zeolites as components of antimicrobial wound covers.

EP 0 837 077 B1 describes antibacterial water- and odor-absorbing compositions for disposable diapers, hygienic tissues, nursing inserts and disposable medical draw sheets which contain at least one particulate water-absorbing resin and at least one antibacterial powder, said antibacterial powder containing at least one particulate inorganic adsorption agent into which at least one antibacterial agent is adsorbed which is selected from C₆₋₈-aliphatic alkyl-quaternary ammonium salt compounds, benzalkonium salt compounds, chlorhexidine compounds and/or polymethylenebiguanidine compounds.

Therefore, there is further a need of a universally applicable wound treating agent which on the one hand promotes blood coagulation (clotting) and on the other hand has an effective sustainable disinfecting activity with little side effects directly at and in the wound.

According to the invention, this object is achieved by a wound treatment agent for humans and animals which contains a zeolite-iodine inclusion compound.

### BRIEF DESCRIPTION OF THE DRAWINGS

The present invention will become more fully understood from the detailed description and the accompanying drawings, wherein:

FIG. 1 is a perspective view of a wound treating article including the wound treating agent of the present invention.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION OF THE PREFERRED EMBODIMENTS

The following description of the preferred embodiment(s) is merely exemplary in nature and is in no way intended to limit the invention, its application, or uses.

The term “zeolite” within the meaning of the present invention represents a widespread group of crystalline silicates, i.e., basically of water-containing alkali and/or alkaline earth alumosilicates of general formula
M_{2/z}O.Al₂O₃.xSiO₂.yH₂O
wherein M represents a mono- or polyvalent metal, especially alkali or alkaline earth cation, H or NH₄, z represents the valence of the cation, x represents a number within a range of from 1.8 to 12, and y represents a number within a range of from 0 to about 8. The stoichiometric ratio of SiO₂ to Al₂O₃ (modulus) is an important characteristic of zeolites. Characteristic of most zeolites is the fact that they release their water continuously and without changing the crystal structures upon heating, take up other compounds instead of the removed water and thus form inclusion compounds, for example, with iodine, from the exchange reaction. Zeolites employed according to the invention contain no or only a small amount of water for the formation of the iodine inclusion compound.

Zeolite is a strong adsorbent, which enables it to bind much water, but it may also bear other materials within its nanopores, for example, iodine. However, since the binding to water is stronger than that to iodine, the iodine will be released in a finely dispersed form and the water bound as soon as the iodine-loaded zeolite comes into contact with water, for example, from blood. This causes immediate hemostasis.

According to the invention, this achieves a synergistic effect between the acceleration of coagulation by water uptake in the zeolite and disinfection by a simultaneous controlled delivery of the iodine, because the iodine-loaded zeolite combines the properties of an iodophor with those of a hemostatic substance. Thus, when applied, the wound is dried and disinfected in one step. The zeolite will absorb the water from the blood, but not the other blood components (except for the salts). Water replaces the iodine adsorbed on the zeolite, which leaves the zeolite. By removing the water, the coagulation factors are enriched to such an extent that the onset of coagulation is clearly expedited.

The wound treating agent according to the invention containing a zeolite-iodine inclusion compound (complex) can be employed, in principle, for wound treatment and the treatment of abrasions and deep wounds, such as cuts, in all living creatures whose blood will spontaneously coagulate, especially in humans and all warm-blooded animals.

In principle, the complex may consist of any zeolite; however, best results are achieved with Ca or Na/Ca zeolites. The zeolite of the zeolite-iodine inclusion compound preferably has a pore size of at least 5 Å. In smaller pore sizes, no stable zeolite-iodine inclusion compound is formed, since the iodine cannot be accommodated in the pores of the zeolite. Such a product contains only little iodine, which is adsorbed superficially.

It is particularly preferred if the zeolite of the zeolite-iodine inclusion compound is loaded with iodine within a range of from 0.1% by weight to its maximum capacity, especially within a range of from 1 to 10% by weight, to achieve efficient disinfection. The iodine is preferably bound physically essentially in the nanopores of the zeolite of the zeolite-iodine inclusion compound.

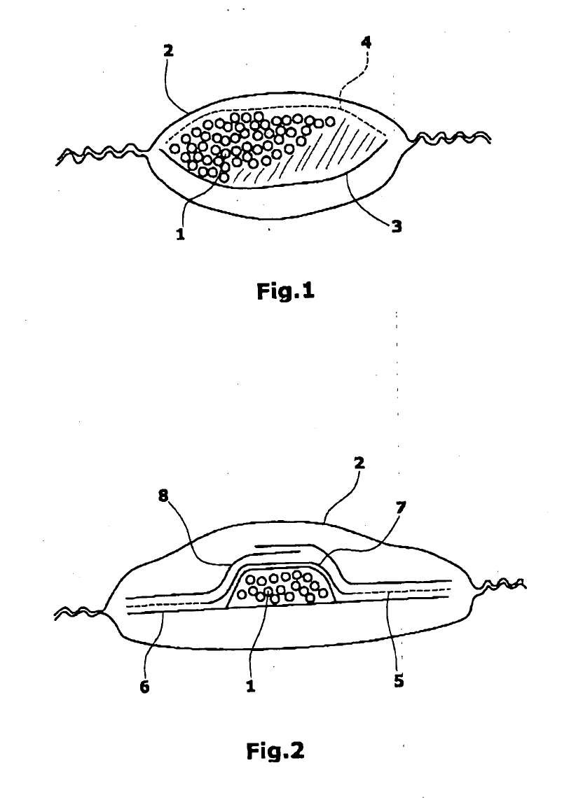

The zeolite-iodine complex may be granulated, pelletized, granular or powdery, the best coagulation results being achieved for an average grain size of the zeolite-iodine inclusion compound of from 0.5 to 2 mm, especially 1 mm. The wound treatment agent may be applied in a loose pad or bag having a porous side, as shown in FIG. 1. The porous side is pressed against the wound. The bag may be removed when the wound is dry, or the felt heat is too high. A typical amount is from 1 to 2 g of zeolite inclusion compound in a loose bag, corresponding to a single dose of iodine of at most 50 mg for the measured release.

The wound treatment agents according to the invention may also contain further compatible auxiliary agents and additives, for example, further wound healing agents or neutral fillers, in addition to the essential components zeolite and iodine.

The article as shown in FIG. 1 includes various components including:

zeolite-iodine inclusion compound (1) (granules),

outer package (2) (outer bag),

solid material (3),

porous material (4) (non-woven fabric etc.), side facing towards the wound,

adhesive layer (5) (skin adhesive),

support fabric (6)

wound pad (7), and

cover paper (8).

For the quick dressing of large-area wounds, one or more of these bags may also be torn open. FIG. 1 shows a corresponding bag in a plastic sheet bag.

The preparation may also be integrated in a self-adhesive wound plaster in small amounts (250 mg). Due to the evolution of heat, thin layers or loose pads should be employed, which can be quickly removed and replaced. FIG. 2 shows a corresponding plaster in a plastic sheet bag.

For the application, it is critical that the product was stored dry before use. In practice, it is indispensable that each portion is individually stored in a water-tight and iodine-tight outer package. For example, a good outer package consists of a relatively tough and thick metal foil or metal-coated sheet, as employed, for example, for suppositories.

Typical situations in which a wound treatment agent according to the invention is applied are those in which a usual plaster would be unfavorable or undesirable, such as with persons or animals who or which are allergic to the adhesive etc. In addition, it is favorable in cases where the bleeding is to be stopped very quickly, or when the wound could be infected, such as for scratches from animals, or for falls in sports, for example, football (soccer) or cycling.

It is particularly preferred that the zeolite-iodine inclusion compound will release about 16% of the theoretical amount of iodine within 20 min in ethanol/water (1:8 v/v) medium at room temperature.

Depending on their intended use, the wound treatment agents according to the invention can additionally contain, for example, synthetic and/or natural polymers, for example, polyacrylates, SIBS compounds, SEBS compounds, natural rubbers, chitosans, alginates, hydrogels, hydrocolloids and/or polyurethanes.

To enhance the disinfecting effect and/or depending on the intended use, they may further contain, in particular, oxidizing inorganic and/or organic compounds, disinfecting chemical compounds, usual fillers and auxiliary agents, perfumes and/or colorants.

According to the invention, the wound treatment agents described are used for the preparation of wound-stilling materials, especially compresses, plasters and/or dressing material.

In a particularly preferred embodiment, the wound treatment agent containing the zeolite-iodine inclusion compound is inserted in a wound compress having a porous surface which is placed on the wound. The compress may be either part of a plaster or loose. Also, the wound treatment agent may be applied to or contained in the plaster in a different way, or more particularly, it may be part of a dressing material.

For large-area injuries, it is also possible to scatter the wound treatment agent according to the invention directly onto the wound area.

This treatment may be effected with humans or animals.

In addition, disposable diapers, sanitary towels or nursing inserts containing the wound treatment agent according to the invention are preferred embodiments of the invention. Wound-stilling materials within the meaning of the invention may further be, for example, pastes or porous or non-porous materials, such as films, for example, siloxane films, polyester films, polyurethane films. However, it is to be taken care that the base of the paste does not contain any materials which are adsorbed by the zeolite.

The wound-stilling material according to the invention is preferably characterized in that the wound treatment agent is covered, on the side facing towards the wound, by a porous textile fibrous material, especially a non-woven or knitted fabric as well as a moisture-permeable sheet which is loaded with from 0.1% to its maximum capacity, preferably from 1 to 10%, of iodine, so that the iodine is bound within the nanopores of the zeolite.

### EXAMPLES

a) Preparation of the Zeolite-Iodine Inclusion Compound

In a desiccator, 150 g of pelletized anhydrous zeolite 5 Å (Aldrich) was weighed. In a separate crystallizing dish, 15 g (10%) of iodine was weighed. The dish was placed into the molecular sieve without allowing direct contact thereof with the iodine. The closed desiccator was evacuated and temperature-controlled to 50° C. The complete transfer of the iodine to the molecular sieve was effected within 2 days.

The same experimental setup was performed with 1% iodine, and in this case, the experiment took only 2 hours. In the alternative performance with an excess of iodine, the molecular sieve had to be placed into the vacuum alone for some hours to free it from superficially adhering iodine.

b) Iodine Release

5 g of zeolite-iodine complex (10% iodine) was admixed with 10 ml of ethanol and 80 ml of demineralized water, and stirred for 20 min. Subsequently, the iodine was titrated with sodium thiosulfate in the usual way. The release of iodine was slow and time-dependent. After 20 min, 16% of the theoretical amount had been released.

c) Microbiological Activity

45 ml of physiological saline was admixed with a germ suspension of Staphylococcus aureus to obtain a concentration of 1.5·10⁷ cfu/ml. To this germ suspension, 1 g of zeolite-iodine complex (10%) was added, followed by slight agitation. After different periods of time, 1 ml each was removed, and the number of surviving germs was determined by a plate-casting method in TSA-agar. Incubation was effected at 37° C. for 24 h.

Sampling after 1 min: strong growth

Sampling after 5 min: no growth

Sampling after 15 min: no growth

Sampling after 30 min: no growth

Thus, it can be established that the zeolite-iodine complex released so much iodine under these conditions that all the germs were killed.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A wound treatment agent which contains a zeolite-iodine inclusion compound.

2. The wound treatment agent according to claim 1, characterized in that the zeolite of said zeolite-iodine inclusion compound has a pore size of at least 5 Å.

3. The wound treatment agent according to either of claims 1 or 2, characterized in that the zeolite of said zeolite-iodine inclusion compound is loaded with iodine within a range of from 0.1% by weight to its maximum capacity.

4. The wound treatment agent according to claim 3, characterized in that the zeolite of said zeolite-iodine inclusion compound is loaded with iodine within a range of from 1 to 10% by weight.

5. The wound treatment agent according to claim 1, characterized in that the iodine is physically bound essentially within the nanopores of the zeolite of said zeolite-iodine inclusion compound.

6. The wound treatment agent according to claim 1, characterized in that said zeolite-iodine inclusion compound is granulated, pelletized or granular.

7. The wound treatment agent according to claim 6, characterized in that said zeolite-iodine inclusion compound has an average grain size of from 0.5 to 2 mm.

8. The wound treatment agent according to claim 1, characterized in that said zeolite-iodine inclusion compound is powdery.

9. The wound treatment agent according to claim 1, characterized in that said zeolite-iodine inclusion compound will release about 16% of the theoretical amount of iodine within 20 min in ethanol/water (1:8 v/v) medium at room temperature.

10. Use of a wound treatment agent according to claim 1 for the preparation of wound-stilling materials.

11. A wound treatment article containing the wound treatment agent of claim 1.

12. A wound treatment article according to claim 11 wherein said article is selected from the group consisting of dressing material, compresses, plasters, disposable diapers, sanitary towels or nursing inserts.

13. A plaster containing the compress according to claim 12.

14. A wound-stilling material characterized in that the wound treatment agent according to claim 1 is covered, on the side facing towards the wound, by a porous textile fibrous material and a moisture-permeable sheet.

15. A wound-stilling material according to claim 14 wherein said porous textile fibrous material is selected from non-woven or knitted fabrics.
